(19) United States
(12) Patent Application Publication
(10) Pub. No.: US 2013/0150444 A1
(43) Pub. Date: Jun. 13, 2013

(54) MODIFIED RELEASE COMPOSITIONS OF MAGNESIUM VALPROATE

(71) Applicant: CADILA HEALTHCARE LIMITED, Ahmedabad (IN)

(72) Inventors: Sunilendu Bhushan ROY, Dist. Ahmedabad (IN); Jay Shantilal KOTHARI, Dist. Ahmedabad (IN); Manish CHAWLA, Dist. Ahmedabad (IN); Mukesh Chimanlal PATEL, Dist. Ahmedabad (IN); Nikunj Arvindbhai LADANI, Dist. Ahmedabad (IN); Umesh Nandkumar KHATAVKAR, Dist. Ahmedabad (IN)

(73) Assignee: CADILA HEALTHCARE LIMITED, Ahmedabad (IN)

(21) Appl. No.: 13/706,906

(22) Filed: Dec. 6, 2012

(30) Foreign Application Priority Data
Dec. 7, 2011 (IN) 3442/MUM/2011

(51) Int. Cl. A61K 47/48; A61K 31/19

(52) U.S. Cl. CPC A61K 47/4823; A61K 31/19; A61K 47/48215
USPC 514/557

(57) ABSTRACT

The present invention relates to a modified release pharmaceutical composition comprising therapeutically effective amounts of magnesium valproate and/or its pharmaceutically acceptable solvates or mixtures thereof and at least one rate controlling polymer. Rate controlling polymer is either hydrophilic or hydrophobic in nature. Mixture of the rate controlling material or more is also used to provide formulations of the present invention.

### FIELD OF THE INVENTION

The present invention relates to pharmaceutical compositions of magnesium valproate and/or its pharmaceutically acceptable solvates. In particular, the invention relates to pharmaceutical compositions comprising magnesium valproate and/or its pharmaceutically acceptable solvates or mixtures thereof in a modified release composition and a process for the preparation thereof.

### BACKGROUND OF THE INVENTION

The Magnesium salt of valproic acid with antiepileptic and potential antineoplastic activities chemically known as magnesium (2+) ion 2-propylpentanoate or magnesium 2-propylvalerate, which has a molecular formula of C₁₆H₃₀MgO₄, and a molecular weight of 310.71. Magnesium valproate possesses pharmacokinetic properties comparable to sodium valproate or valproic acid, it dissociates in the gastrointestinal tract and is absorbed into the circulation as magnesium ions and valproic acid ions; and has important advantages in comparison with either sodium valproate or valproic acid. Among the therapeutic advantages of magnesium valproate are the clinical observations that magnesium valproate exhibits a slower and more regular absorption rate, which prevents the variations in plasma levels of valproate typically observed when sodium salts of valproic acid are administered. Additional therapeutic benefits are afforded by magnesium ions, which possess anticonvulsant and sedative properties. [Rabasseda, Drugs of Today, Vol. 31, No. 3, 1995, pp. 185-190.]

ES430062 discloses method for the preparation of Magnesium dipropylacetate [(Pr2CHCO2−)2 Mg2+], which has antiepileptic activity, is obtained by treating dipropylacetic acid with magnesium oxide in alcoholic medium and isolating the product by filtration, concentration, preparation, recrystallization, and drying. The method has the following limitations viz., reaction is carried out in a suspension and lengthy reaction time, final product is contaminated with un-reacted magnesium oxide, product is an amorphous solid that is difficult to purify and dry.

U.S. Pat. No. 5,180,850 discloses a new crystalline form of magnesium valproate obtained by reacting valproic acid with a substantially stoichiometric amount of magnesium alkoxides selected from magnesium ethoxide, magnesium propoxide, and magnesium isopropoxide in alcoholic solution. The magnesium salt of valproic acid is isolated in a microcrystalline form by solvent evaporation or by acetone precipitation. The new crystalline form is endowed with enhanced bioavailability and may be used in a particularly pure form to prepare pharmaceutical compositions which are useful for treating Central Nervous System pathologies. However, the methods disclosed above have following shortcomings viz., Product isolation by solvent evaporation provides product that is contaminated by incompletely reacted starting materials or adventitious contaminants in starting materials or solvents. When the reaction is carried out in ethanol, the quantity of magnesium ethoxide specified is not completely dissolved in the volume of ethanol.

EP0956010 discloses pharmaceutical microspheres of valproic acid for oral administration. The invention concerns pharmaceutical microspheres containing as active principle a mixture of valproic acid and of its salts, associated with a matrix support selected from glycerol esters, hydrogenated oils, esterified polyethylene glycols, waxes and their mixtures. Thus, microspheres prepared according to this invention contain beeswax 68.42%, valproic acid 9.6%, and sodium valproate 22%.

CN1302607 discloses a sustained-release magnesium propylvalerate tablet and its preparation. A sustained-release tablet of Mg propylvalerate is prepared from Mg propylvalerate 20-30, HPMC 20-50, Et cellulose 0.5-8, stearic acid 0-5, ethanol 0-30, and Mg stearate 0-3%. The sustained-release tablet is an antiepileptic medicine.

U.S. Pat. No. 7,482,486 discloses methods for the preparation and formulation of magnesium valproate hydrate. The present invention relates to methods for preparing magnesium valproate hydrate and administering this compound to a subject in need of treatment with valproate. Pharmaceutical compositions are also provided that are useful therapies for the treatment of neurological, immunological, and viral-mediated disorders in warm-blooded mammals. The shortcomings associated with the method disclosed above are complete dissolution of the magnesium ethoxide solid in ethanol was never achieved, and yields of the final product, magnesium valproate, was obtained is 66% only.

The Magnesium salt of valproate is less frequently used in the pharmaceutical formulations even though of its therapeutic and clinical benefits. The reason associated with the above discussed shortcomings is a difficulty in preparing the magnesium salt of valproic acid with physicochemical properties apposite to pharmaceutical formulations and applications. The above discussed methods suffer from deficiencies in pharmaceutical manufacturing utility and provide magnesium valproate lacking the purity, solubility, and physicochemical properties which enable its use in pharmaceutical formulations. Therefore, there exists a continuous need for finding suitable ways to overcome the problem of pharmaceutical manufacturing utility and physicochemical properties apposite to pharmaceutical formulations of magnesium valproate.

Hence, there is a need to develop a modified release formulation of magnesium valproate and/or its pharmaceutically acceptable solvates or mixtures thereof and a process for the preparation the same.

### SUMMARY OF THE INVENTION

The present invention relates to modified release pharmaceutical compositions of Magnesium valproate and/or its pharmaceutically acceptable solvates or mixtures thereof.

In one aspect the present invention provides a modified release pharmaceutical composition comprising magnesium valproate or its pharmaceutically acceptable solvates, one or more rate controlling polymers and one or more pharmaceutically acceptable excipients.

In another aspect the present invention provides rate controlling polymers comprises one or more of hydrophilic polymers or hydrophobic polymers optionally an enteric polymer or mixtures thereof.

In another aspect the present invention provides a modified release pharmaceutical composition comprising magnesium valproate and/or its pharmaceutically acceptable solvates or mixtures thereof, one or more rate controlling polymers and one or more pharmaceutically acceptable excipients, wherein the composition when measured in type II dissolution apparatus, paddle, at 100 rpm, at a temperature of 37±0.5° C., in 500 ml of 0.1N HCl for 45 minutes, followed by 900 ml of 0.05M phosphate buffer containing 75 Mm sodium lauryl sulfate, pH 5.5, for remainder of testing period exhibit an in-vitro dissolution profile as follows:
i. no more than 40% of the total valproate is released after 3 hours of measurement in said apparatus;
ii. from about 40 to about 75% of total valproate is released after 6 hours of measurement in said apparatus;
iii. from about 60 to about 85% of total valproate is released after 12 hours of measurement in said apparatus; and
iv. more than 90% of the total valproate is released after 18 hours of measurement in said apparatus.

### DETAILED DESCRIPTION OF THE INVENTION

Accordingly, the present invention provides modified release pharmaceutical compositions comprising therapeutically effective amounts of magnesium valproate or its pharmaceutically acceptable solvates, one or more rate controlling polymers and one or more pharmaceutically acceptable excipients.

As used herein the term “magnesium valproate” is not particularly limited to the base or salt and includes anhydrates, solvates, as well as crystalline and amorphous forms of magnesium valproate.

According to the present invention, magnesium valproate can be used in therapeutic effective amount in patient need thereof.

Present invention provides a modified release formulations of magnesium valproate comprising magnesium valproate and at least one rate-controlling polymer. Rate controlling polymer is either hydrophilic or hydrophobic in nature. Mixture of the rate controlling material or more is also used to provide formulations of present invention.

The modified release formulation of the present invention is in the form of a matrix comprising magnesium valproate, one or more hydrophilic polymers, optionally an enteric polymer and pharmaceutically acceptable excipients. The formulations of the present invention can be in the form of oral dosage forms such as tablets, capsules, pellets, granules, microtablets etc. Preferably, formulation is either compressed into tablets or granulated or coated with suitable excipients and filled into capsules.

Magnesium valproate used in the present invention can be in the range of 10 to 80% weight of the composition and the rate controlling polymer used in the present invention can be in the range of 5 to 70% weight of the composition.

Suitable hydrophilic polymers include polyethylene oxides, cellulose ethers such as hydroxypropyl methyl cellulose, hydroxypropyl ethyl cellulose, hydroxy ethylcellulose, hydroxypropylcellulose, or other water soluble or swellable polymers such as sodium carboxymethyl cellulose, locust bean gum, xanthan gum, acacia, tragacanth gum, guar gum, karaya gum, alginates, gelatin, albumin, carbomers, alginates, polyvinyl pyrollidones or mixtures thereof. These hydrophilic polymers also include polyacrylate polymers, such as homopolymers based on acrylic acid cross-linked with allyl sucrose or allyl pentaerythritol, or copolymers based on acrylic acid and long chain (C₁₀-C₃₀) allyl acrylates cross-linked with allylpentaerythritol. The polyacrylate polymers may be used alone or in admixture with cellulose ethers such as methylcellulose, hydroxypropyl methylcellulose, hydroxypropylcellulose, hydroxyethylcellulose, and the like. According to the invention, the hydrophilic polymers are present in amounts ranging from about 5% to about 70% by weight of the system.

The enteric polymers that may be used in the present invention include but are not limited to polyacrylate copolymers such as Methacrylic Acid Copolymer, cellulose derivatives, such as cellulose acetate phthalate, hydroxypropyl methylcellulose phthalate, hydroxypropyl methylcellulose acetate succinate; and polyvinyl acetate phthalate and the like Zein/shellac etc. also be included. Preferably enteric polymer may be Polymethacrylate. Polymethacrylate can be a cationic, anionic or neutral polymer or copolymer on the basis of monomers having a methacrylic moiety. Mixtures of such polymers or copolymers can also be used. Preferably, the polymethacrylate is a polymer or copolymer based on at least one of dimethylaminoethyl methacrylates, methacrylic acid and methacrylic acid esters. It is particularly preferred that the polymethacrylate is a methacrylic acid copolymer or a copolymer of dimethylaminoethyl methacrylates and methacrylic acid esters. Various different types of polymethacrylates are commercially available e.g. under the trade name Eudragit. The enteric polymer may be present in amounts 0.5% to 50% by weight of the system.

In the present invention, magnesium valproate is dispersed in hydrophilic polymers and the release of the drug is controlled primarily by diffusion of the drug, or by surface erosion of the hydrophilic polymers into the surrounding medium, or by a combination of the two processes achieved by the methods known to the person skilled in the art.

According to another aspect of the present invention, a modified release formulations comprising magnesium valproate, hydrophobic rate-controlling polymer alone or mixture of hydrophobic and hydrophilic rate-controlling polymer. Hydrophobic rate-controlling polymer is either incorporated into or coated onto dosage form to provide modified release formulations.

The most preferred hydrophobic rate-controlling polymer is selected from ethyl cellulose, polymethacrylates, and mixtures thereof. Most preferred polymethacrylates are poly ethyl acrylate, poly methyl methacrylate, poly trimethylaminoethyl methacrylates, Poly vinyl acetate, and mixtures thereof. Other suitable hydrophobic polymers include polymers and/or copolymers derived from acrylic or methacrylic acid and their respective esters, zein, waxes, shellac and hydrogenated vegetable oils.

Modified release formulation of magnesium valproate further comprises pharmaceutically acceptable excipients. Pharmaceutically acceptable excipients includes and can be selected from any of disintegrants, diluents, stabilizers, binder, lubricants, glidants, coloring agents, stabilizers, masking agents, surfactants, solubilizers, moisture scavengers, antioxidants, buffering agent, adsorbents, adhering agents and the like.

Disintegrants can be selected from calcium phosphate, (mono/di/tri basic), carboxy methyl cellulose salt such as calcium, sodium; cellulose, microcrystalline cellulose, chitosan, silicone dioxide, croscarmellose sodium, Cross-linked polyvinyl pyrrolidone, guar gum, L-hydroxypropyl cellulose, magnesium aluminum silicate, methyl cellulose, povidone, sodium starch glycolate, starch or mixtures thereof or the well-known disintegrants known by the person skilled in the art.

Diluents can be selected from calcium carbonate, calcium phosphate, calcium sulfate, cellulose, cellulose acetate, dextrates, dextrines, dextrose, ethyl cellulose, microcrystalline cellulose, polydextrose, polymethacrylates, sucrose, lactose, starch, mannintol or mixtures thereof or the well-known diluents known by the person skilled in the art.

Binder can be selected from acacia, alginic acid, carbomers, carboxymethyl cellulose sodium, chitosan, dextrates, dextrins, dextrose, ethyl cellulose, gelatin, glucose, guar gum, hydroxyethyl cellulose, hydroxyethylmethyl cellulose, hydroxy propyl cellulose, hydroxypropyl methyl cellulose, hypromellose, magnesium silicate, aluminium silicate, maltodextrin, maltose, micro crystalline cellulose, poloxamer, polydextrose, polyethylene oxide, povidone, sodium alginate, starch, sucrose or mixtures thereof or the well-known binder known by the person skilled in the art.

Stabilizers can be selected from sodium citrate, sodium chloride, dipotassium phosphate, meglumine, sodium ascorbate, potassium chloride, sodium sulfite, Poloxamer 188/407, Polyethylene glycol, glyceryl monooleate, alginic acid, albumin, ammonium alginate, ascorbic acid, ascorbyl palmitate, bentonite, butylated hydroxytolune, calcium alginate, calcium state, carboxymethylcellulose calcium, carboxymethylcellulose sodium, carrageenan, ceratonia, colloidal silicon dioxide, cyclodextrins, diethanolamine, edetates, ethylene glycol palmisterate, glycerin monosterate, guargum, magnesium aluminium silicate, lecithin, hypromellose, hydroxy propyl cellulose, polacrilin potassium, pectin, poloxamer, polyvinyl alcohol, propyl gallate, propylene glycol, xylitol, zinc acetate, raffinose, sodium borate, trehalose, propylene glycol alginate, sulfobutylether beta-cyclo dextrin or mixtures thereof or the well-known stabilizers know by the person skilled in the art.

Glidants can be selected from the group consisting of silicon dioxide, talc, calcium stearate, Magnesium stearate and aluminium stearate, or mixtures thereof. Lubricants can be selected from magnesium stearate, magnesium lauryl sulfate, sodium lauryl sulfate, sodium stearyl fumarate, calcium stearate, microcrystalline cellulose and silicone dioxide, hydrogenated cottonseed oil, hydrogenated castor oil, stearic acid, zinc stearate or mixtures thereof.

Buffering agents can be selected from ammonia solution, calcium carbonate, calcium phosphate, citric acid, sodium phosphate, diethanol amine, malic acid, monosodium glutamate, phosphoric acid, potassium citrate, sodium acetate, sodium bicarbonate, sodium borate, sodium citrate, sodium hydroxide, sodium lactate, triethanol amine or mixtures thereof or the well-known buffering agent known by the person skilled in the art.

The modified release formulation of magnesium valproate can be prepared by employing any method selected from particle coating, wet granulation, direct compression, dry granulation, melt granulation, monolithic or reservoir or combination of monolithic and reservoir technology or the method known by the person skilled in the art.

A modified release pharmaceutical composition comprising magnesium valproate and/or its pharmaceutically acceptable solvates or mixtures thereof, one or more rate controlling polymers and one or more pharmaceutically acceptable excipients, wherein the composition when measured in type II dissolution apparatus, paddle, at 100 rpm, at a temperature of 37±0.5° C., in 500 ml of 0.1N HCl for 45 minutes, followed by 900 ml of 0.05M phosphate buffer containing 75 Mm sodium lauryl sulfate, pH 5.5, for remainder of testing period exhibit an in-vitro dissolution profile as follows:
i. no more than 40% of the total valproate is released after 3 hours of measurement in said apparatus;
ii. from about 40 to about 75% of total valproate is released after 6 hours of measurement in said apparatus;
iii. from about 60 to about 85% of total valproate is released after 12 hours of measurement in said apparatus; and
iv. more than 90% of the total valproate is released after 18 hours of measurement in said apparatus.

A modified release pharmaceutical composition comprising magnesium valproate and/or its pharmaceutically acceptable solvates or mixtures thereof, one or more rate controlling polymers and one or more pharmaceutically acceptable excipients, wherein said composition retains potency of at least 90% after 6 m at 40° C., 75% RH.

The modified release formulation of magnesium valproate and its process described in the present invention is demonstrated in examples illustrated below. These examples are provided as illustration only however, in no way these examples should be construed as limiting the scope of the invention.

### EXAMPLES

### Example 1

Magnesium valproate, silicon dioxide, high viscosity hypromellose, low viscosity hypromellose, high viscosity hydroxypropyl Cellulose and half the quantity of low viscosity hydroxypropyl Cellulose was shifted through 20 mesh sieves and mixed in rapid mixer granulator. Half quantity of low viscosity Hydroxypropyl Cellulose was dissolved in solvent mixture of ethanol and dichloromethane and granulated the above contents. Dried the wet mass and dry size of the granules. Silicondioxide and talc was sifted through 60 mesh sieve and mixed with the dried granules. Magnesium Stearate was sifted through 60 mesh sieve and lubricated the granules. Lubricated granules were compressed into tablets and coated the core matrix tablets using Opadry White.

| S. No. | Ingredients | Qty/tab (mg) |
|---|---|---|
| 1 | Magnesium valproate equivalent to Valproic Acid | 538.64 |
| 2 | Silicon Dioxide | 9.00 |
| 3 | High viscosity Hypromellose | 456.00 |
| 4 | Low viscosity Hypromellose | 84.00 |
| 5 | High viscosity Hydroxypropyl Cellulose | 162.00 |
| 6 | Low viscosity Hydroxypropyl Cellulose | 66.00 |
| 7 | Silicon Dioxide | 34.36 |
| 8 | Talc | 4.00 |
| 9 | Mg. stearate | 14.00 |
| 10 | Opadry white | 41.00 |
| | Total | 1409.000 |

### Example 2

Magnesium valproate, microcrystalline cellulose, high viscosity hypromellose, low viscosity hypromellose, high viscosity hydroxypropyl cellulose and low viscosity hydroxypropyl Cellulose was shifted through 20 mesh sieve and mixed in rapid mixer granulator. Half quantity of low viscosity hydroxypropyl cellulose was dissolved in solvent mixture of ethanol and dichloromethane and granulated the above contents. The wet mass was dried and dry size of the granules. Colloidal silicondioxide and magnesium stearate was sifted through 60 mesh sieve and mixed with granules.

Magnesium Stearate was sifted through 60 mesh sieve and lubricated granules. Lubricated granules were compressed into tablets and coated the core matrix tablets using Opadry White.

| S. No. | Ingredients | Qty/tab(mg) |
|---|---|---|
| 1 | Magnesium valproate equivalent to Valproic Acid | 538.64 |
| 2 | Microcrystalline Cellulose | 40.36 |
| 3 | High viscosity Hypromellose | 353.00 |
| 4 | Low viscosity Hypromellose | 125.00 |
| 5 | High viscosity Hydroxypropyl Cellulose | 120.00 |
| 6 | Low viscosity Hydroxypropyl Cellulose | 35.00 |
| 7 | Low viscosity Hydroxypropyl Cellulose | 60.00 |
| 8 | Colloidal Silicon Dioxide | 7.00 |
| 9 | Mg. stearate | 11.00 |
| 10 | Opadry white | 30.00 |
| | Total | 1320.00 |

### Example 3

Magnesium valproate, lactose monohydrate, high viscosity hypromellose, was shifted through 20 mesh sieve and mixed in rapid mixer granulator. Low viscosity Hypromellose was dissolved in solvent mixture of ethanol and dichloromethane and granulated the contents. The wet mass was dried in dryer and dry size of the granules. Colloidal Silicon Dioxide and Microcrystalline cellulose was sifted through 40 mesh sieves and mixed with granules. The lubricated granules were compressed into tablets and coated the core matrix tablets using Opadry White.

| S. No. | Ingredients | Qty/tab (mg) |
|---|---|---|
| 1 | Magnesium valproate equivalent to Valproic Acid | 538.64 |
| 2 | Lactose Monohydrate | 48.86 |
| 3 | High viscosity Hypromellose | 300.00 |
| 4 | Low viscosity Hypromellose | 50.00 |
| 5 | Colloidal Silicon Dioxide | 20.00 |
| 6 | Microcryastalline Cellulose | 32.50 |
| 7 | Mg. stearate | 10.00 |
| 8 | Opadry white | 30.00 |
| | Total | 1030.00 |

### Example 4

Magnesium Valproate, Lactose Monohydrate, high viscosity Hypromellose, was sifted through 20 mesh sieve and mixed in rapid mixer granulator. Low viscosity Hypromellose was dissolved in solvent mixture of ethanol and dichloromethane and granulated the above contents. The wet mass was dried in dryer dry size the granules. Colloidal silicon dioxide and microcrystalline cellulose was sifted through 40 mesh sieve and mixed with granules. The lubricated granules were compressed into tablets. Ethyl Cellulose and Low viscosity Hydroxypropyl cellulose was dissolved in solvent mixture of ethanol and dichloromethane and compressed matrix tablets was coated.

| S. No. | Ingredients | Qty/tab (mg) |
|---|---|---|
| 1. | Magnesium valproate equivalent to Valproic Acid | 538.64 |
| 2. | Lactose Monohydrate | 48.86 |
| 3. | High viscosity Hypromellose | 300.00 |
| 4. | Low viscosity Hypromellose | 50.00 |
| 5. | Colloidal Silicon Dioxide | 20.00 |
| 6. | Microcryastalline Cellulose | 32.50 |
| 7. | Mg. stearate | 10.00 |
| 8. | Ethyl cellulose | 25.00 |
| 9. | Low viscosity Hydroxypropyl cellulose | 25.00 |
| | Total | 1050 |

### Example 5

Magnesium Valproate, Lactose Monohydrate, Polyethylene oxide, was shifted through 20 mesh sieve and mixed in rapid mixer granulator. Low viscosity Hypromellose was dissolved in solvent mixture of ethanol and dichloromethane and granulated the above contents. The wet mass was dried in dryer and dry size the granules. Colloidal Silicon Dioxide and Microcrystalline cellulose was sifted through 40 mesh sieve and mixed with granules. The lubricated granules were compressed into tablets and coated the core matrix tablets using Opadry White.

| Sr. No. | Ingredients | Qty/tab (mg) |
|---|---|---|
| 1. | Magnesium valproate equivalent to Valproic Acid | 538.64 |
| 2. | Microcryastalline Cellulose | 48.86 |
| 3. | Polyethylene Oxide | 353.00 |
| 4. | Hypromellose (Methocel E5) | 125.00 |
| 5. | Colloidal Silicon Dioxide | 20.00 |
| 6. | Microcryastalline Cellulose | 32.50 |
| 7. | Mg. stearate | 10.00 |
| 8. | Opadry white | 30.00 |
| | Total | 1030.00 |

### Example 6

When 500 mg of magnesium valproate ER tablet of the present according to the above examples measured in type II dissolution apparatus, paddle, at 100 rpm, at a temperature of 37±0.5° C., in 500 ml of 0.1N HCl for 45 minutes, followed by 900 ml of 0.05M phosphate buffer containing 75 Mm sodium lauryl sulfate, pH 5.5, for remainder of testing period exhibit an in-vitro dissolution profile as follows:

| Media | 0.1N HCl followed by pH 5.5 Phosphate | |
|---|---|---|
| | buffer with 75 mM SLS Sinker | |
| Volume | 900 | ml |
| Apparatus | II Paddle | |
| Agitation | 100 | rpm |

Pack: 100 CC Heavy wt. with 2 gm silica gel canister
% Release

| | | 3M 40° C./ | 6M 40° C./ |
|---|---|---|---|
| | Initial | 75% RH | 75% RH |
| Time (hr) | | | |
| 0 | 0 | 0 | 0 |
| 0.75 | 2.70 | 3.1 | 2.50 |
| 1.5 | 20.80 | 20.5 | 19.70 |
| 3 | 33.40 | 33.3 | 32.50 |
| 6 | 53.50 | 52.5 | 53.10 |
| 9 | 66.90 | 67.5 | 68.20 |
| 12 | 80.60 | 79.6 | 80.10 |
| 15 | 89.70 | 88.8 | 90.40 |
| 18 | 95.80 | 95.4 | 97.70 |
| 21 | 101.30 | 96.1 | 101.10 |
| 24 | 100.60 | 95.7 | 101.60 |
| Assay | 100.5 | 100.3 | 99.6 |
| Related Substances | | | |
| Methyl Ester | ND | ND | ND |
| Valeric acid | 0.01% | 0.01% | 0.01% |
| 2 Methyl Valeric acid | ND | ND | ND |
| 2 Ethyl Valeric acid | 0.07% | 0.08% | 0.07% |
| 2 Iso Propyl Valeric acid | 0.04% | 0.05% | 0.04% |
| 2 Butyl Valeric acid | ND | 0.00% | ND |
| single Max Unknown | 0.01% | 0.01% | 0.01% |
| Totoa Impurity | 0.15% | 0.17% | 0.15% |
| Water by KF | 4.67% | 4.05% | 4.16% |

The comparative dissolution study was performed between innovator sample and the examples of the present invention and under the below mentioned conditions and the results of the study was illustrated in the table 1:

Media: 0.1N HCl 45 min followed by 5.5 phosphate buffer 5 mM Sodium dodecyl sulfate.

Volume: Acid stage-500 ml Buffer stage-900 ml.

Apparatus: Paddle, 100 RPM

TABLE 1
comparative dissolution data

| Time (Hrs) | Innovator | Example 1 | Example 2 | Example 3 |
|---|---|---|---|---|
| 0.75 | 3.83 | 2.10 | 1.40 | 3.70 |
| 1.5 | 19.10 | 16.70 | 19.40 | 22.50 |
| 3 | 30.97 | 28.30 | 34.00 | 35.10 |
| 6 | 53.97 | 44.70 | 54.80 | 56.30 |
| 9 | 69.80 | 58.30 | 70.00 | 72.70 |
| 12 | 90.83 | 70.10 | 82.10 | 84.50 |
| 15 | 100.47 | 80.10 | 90.10 | 92.80 |
| 18 | 103.07 | 88.10 | 94.50 | 96.40 |
| 21 | 100.33 | 92.90 | 95.90 | 96.50 |
| 24 | 103.17 | 94.90 | 95.70 | 96.30 |

While the invention has been described in terms of its specific embodiments, certain modifications and equivalents will be apparent to those skilled in the art and are intended to be included within the scope of the invention.

We claim:

1. A modified release pharmaceutical composition comprising magnesium valproate or its pharmaceutically acceptable solvates, one or more rate controlling polymers and one or more pharmaceutically acceptable excipients.

2. The composition as claimed in claim 1, wherein the composition comprises from about 10% to about 80% w/w of magnesium valproate or its pharmaceutically acceptable solvates.

3. The composition as claimed in claim 1, wherein the rate controlling polymers comprise one or more of hydrophilic polymers, or hydrophobic polymers, optionally an enteric polymer or mixtures thereof.

4. The composition as claimed in claim 1, wherein the rate controlling polymers comprises from about 5 to about 70% w/w of the composition.

5. The composition as claimed in claim 3, wherein the hydrophilic polymers comprise one or more of polyethylene oxides, cellulose ethers, polyacrylate polymers, or mixtures thereof or other water soluble or swellable polymers.

6. The composition as claimed in claim 5, wherein the cellulose ethers comprise one or more of methylcellulose, hydroxypropyl methyl cellulose, hydroxypropyl ethyl cellulose, hydroxy ethylcellulose, hydroxypropylcellulose, or mixtures thereof.

7. The composition as claimed in claim 5, wherein the water soluble or swellable polymers comprise one or more of sodium carboxymethyl cellulose, locust bean gum, xanthan gum, acacia, tragacanth gum, guar gum, karaya gum, alginates, gelatin, albumin, carbomers, alginates, and polyvinyl pyrollidones.

8. The composition as claimed in claim 5, wherein the polyacrylate polymers comprise one or more of homopolymers based on acrylic acid cross-linked with allyl sucrose or allyl pentaerythritol, or copolymers based on acrylic acid and long chain (C₁₀-C₃₀) allyl acrylates cross-linked with allylpentaerythritol.

9. The composition as claimed in claim 3, wherein the hydrophobic polymer comprises one or more of ethyl cellulose, polymethacrylates, polymers and/or copolymers derived from acrylic or methacrylic acid and their respective esters, zein, waxes, shellac and hydrogenated vegetable oils and mixtures thereof.

10. The composition as claimed in claim 3, wherein the enteric polymers comprises one or more of polyacrylate copolymers such as methacrylic acid copolymer, cellulose derivatives, such as cellulose acetate phthalate, hydroxypropyl methylcellulose phthalate, hydroxypropyl methylcellulose acetate succinate; and polyvinyl acetate phthalate, and the mixtures thereof.

11. The composition as claimed in claim 1, wherein the pharmaceutically acceptable excipients comprises disintegrants, diluents, stabilizers, binder, lubricants, glidants, coloring agents, stabilizers, masking agents, surfactants, solubilizers, moisture scavengers, antioxidants, buffering agent, adsorbents, and adhering agents.

12. A modified release pharmaceutical composition comprising magnesium valproate and/or its pharmaceutically acceptable solvates or mixtures thereof, one or more rate controlling polymers and one or more pharmaceutically acceptable excipients, wherein the composition when measured in type II dissolution apparatus, paddle, at 100 rpm, at a temperature of 37±0.5° C., in 500 ml of 0.1N HCl for 45 minutes, followed by 900 ml of 0.05M phosphate buffer containing 75 Mm sodium lauryl sulfate, pH 5.5, for remainder of testing period exhibit an in-vitro dissolution profile as follows:
i) no more than 40% of the total valproate is released after 3 hours of measurement in said apparatus;
ii) from about 40 to about 75% of total valproate is released after 6 hours of measurement in said apparatus;
iii) from about 60 to about 85% of total valproate is released after 12 hours of measurement in said apparatus; and
iv) more than 90% of the total valproate is released after 18 hours of measurement in said apparatus.

13. A modified release pharmaceutical composition comprising magnesium valproate and/or its pharmaceutically acceptable solvates or mixtures thereof, one or more rate controlling polymers and one or more pharmaceutically acceptable excipients, wherein said composition retains potency of at least 90% after 6 m at 40° C., 75% RH.
